(19) United States
(12) Patent Application Publication
(10) Pub. No.: US 2018/0031517 A1
(43) Pub. Date: Feb. 1, 2018

(54) Devices, Systems and Methods for Use of Electrical Ion Sensors Based on Metal-Organic Supercontainers

(71) Applicant: South Dakota Board of Regents, Pierre, SD (US)

(72) Inventors: Nathan Netzer, Uppsala (SE); Indrek Must, Voibla (EE); Yupu Qiao, Vermillion, SD (US); Zhenqiang Wang, Vermillion, SD (US); Zhen Zhang, Sollentuna (SE); Shi-Li Zhang, Stockholm (SE)

(21) Appl. No.: 15/665,370

(22) Filed: Jul. 31, 2017

Related U.S. Application Data
(60) Provisional application No. 62/368,821, filed on Jul. 29, 2016.

(51) Int. Cl. G01N 27/414; G01N 33/18

(52) U.S. Cl. CPC G01N 27/414; G01N 33/1813

(57) ABSTRACT

The disclosed apparatus, systems and methods relate to the use of metal-organic supercontainers as a size-selective ionophore by incorporation into a substrate for ion sensing electrodes.

### CROSS REFERENCE TO RELATED APPLICATIONS

This application claims priority to the U.S. Provisional Application No. 62/368,821 filed Jul. 29, 2016 and entitled “Devices, Systems and Methods for Use of Electrical Ion Sensors Based on Metal-Organic Supercontainers” which is hereby incorporated by reference in its entirety under 35 U.S.C. § 119(e).

### GOVERNMENT SUPPORT

This invention was made with government support under awards CHE 1352279 and DMR 1709912, awarded by the US National Science Foundation. The government has certain rights in the invention.

### TECHNICAL FIELD

The disclosed technology relates generally to container molecules and in particular, to the systems, devices, methods, and design principles allowing the use of metal-organic supercontainers as a size-selective ionophore by incorporation into a mixed-matrix membrane for ion-selective electrodes. This has implications in a variety of industries in which there is a demand for rapid and efficient monitoring of molecular ions.

### BACKGROUND

The disclosure relates to the systems, devices, methods, and design principles allowing for the use of metal-organic supercontainers (MOSCs) as a size-selective ionophore. In various implementations, the MOSCs are incorporated into a substrate such as a mixed-matrix membrane (MMM) for ion-selective electrodes (ISEs). In various implementations, ion sensing—such as electrical ion sensing—has a variety of applications, including, but certainly not limited to, water quality and environmental control and the detection of charged species in electrolytes for chemical, biological, and medical monitoring.

Currently, one method of forming MMMs is to incorporate ion-binding receptors, also known as “ionophores,” with ion-exchange sites into a polymer matrix, allowing for mechanical stability and increased ion-selectivity. Another method incorporates charged ionophores without ion-exchange sites. Currently there are a variety of commercially available ionophores and protocols for fabricating ISEs with specific binding affinities to about 30 cations and anions.

While supramolecular host materials such as metal-organic frameworks (MOFs) have become popular in the electrical ion sensing field because they have favorable binding sites, MOFs are inherently insoluble in most common solvents and thus lack certain desirable characteristics needed for incorporation into ISEs, such as solution processability.

Thus, there is a need in the art for technologies that allow for rapid and precise monitoring of molecular ions for both environmental and biomedical applications.

### BRIEF SUMMARY

To facilitate understanding of the disclosure, as used herein, the term “solid-liquid junction sensing interface” may be used to reference various embodiments of a sensing interface. In certain implementations, an MOSC is bound directly to a solid-contact. In further implementations a junction is formed between organic (MMM) and aqueous phases. For simplicity and brevity, and as would be understood, the MMM of the second definition may be referred to herein as a solid although it is truly an organic (solvent) phase with a phase transition.

In the various implementations, the ion sensing technology disclosed herein offers a significant improvement over the ion sensing technology found in the prior art, which does not have the capacity to accurately measure large molecular ions. Data provided herein demonstrates the ability of the disclosed system, devices and methods to accurately measure large molecular ions with an ISE using a MOSC incorporated into a substrate or scaffolding.

In certain embodiments, the disclosed is an improved apparatus for detection of molecular ions. The apparatus is designed to incorporate MOSCs into a substrate, which allows the MOSCs to selectively bind the desired molecular ion/s for accurate measurement using an ISE. The apparatus improves on the ion sensing capability of previously developed ISEs with incorporation of MOSC into the device, system or method.

According to certain embodiments, the ion sensing technology disclosed herein may be used to conduct analysis of water, sweat, blood, saliva and many other liquid samples. The liquid sample may consist of a complex matrix of small molecules, molecular ions, and elemental ions. Analysis of these samples can yield information regarding water contamination, an individual's physiological state or early disease diagnosis. For example, the glucose level in human sweat is closely correlated to the blood glucose level. The sweat lactate is potentially a very useful early indicator of pressure ischemia. Neurochemicals, as another example, which include neurotransmitters and important elemental ions are actively involved in cell growth, replication, response, and communication in the neuronal network.

Potentiometric sensors employing ion-binding receptors (ionophores) have been extensively studied in the prior art for selective detection of cations and anions. The prior art ionophores are mostly limited to detection of elemental and other small inorganic ions. The presently disclosed use of MOSCs as ionophores improves on the prior art in the ability to detect large molecular ions without resorting to high performance liquid chromatography and/or gas chromatography-spectrometry which require highly trained operators and expensive, bulky instrumentation. The unique structure of MOSCs creates chemically tunable exo- and endo-cavities enabling the design of novel ionophores for highly specific ion detection.

In various implementations, the device demonstrates a near-Nernstian response that allows for very accurate concentration measurements.

In various implementations, the device demonstrates many positive qualities including the lack of any observable leeching, shown in Example 2, and stability in a wide range of environments, shown in Example 3.

In certain implementations, the device can measure electrical response by using an ion sensing electrode constructed on the gate terminal of an ISFET, shown in Example 4.

One Example includes a chemically tunable metal-organic system including: a supercontainer including one or more of: an exo-cavity, an endo-cavity and a substrate, where the substrate is combined with the supercontainer to form a scaffold, and a reference probe, where the system is constructed and arranged to detect ions. Implementations of this Example may include one or more of the following features. The system where the system detects ions by measuring an electrical response. The system where the substrate includes: a mixed-matrix membrane, or a solid sensing surface. The system where the mixed-matrix membrane is a polymer matrix. The system where the solid sensing surface is selected from the group including of oxide, nitride, and metal. The system where the system measures electrical response using a solid-contact ion sensing electrode. The system where the system measures electrical response using an ion-sensing electrode with an inner filling solution and an internal Ag/AgCl reference electrode. The system where the system measures electrical response using an ion-sensing electrode constructed on an ion-sensitive field-effect transistor gate terminal. The device where the substrate is a mixed matrix membrane. The device where the mixed matrix membrane includes a variety of polymers. The device where the polymer is selected from a group including of polyvinyl chloride, polydimethylsiloxane, poly(methyl methacrylate), urushi, cellulose triacetate, poly aniline, poly urethane, siloprene, poly-(vinyl chloride-co-vinyl acetate-co-hydroxypropyl acrylate), and poly(vinyl chloride-co-vinyl acetate-co-vinyl alcohol). The device where the substrate is a solid sensing surface. The device where the solid sensing surface is selected from a group including of oxide, nitride, and metal. The device further including an ion-sensitive field-effect transistor. The system where the ion sensing electrode is a solid contact electrode and the substrate is placed on the electrode. The system where the system is placed on an ion-sensitive field-effect transistor gate terminal to measure drain current change of the ion-sensitive field-effect transistor. The system where the substrate is incorporated into the ion sensing electrode. The system where the plurality of metal organic super containers are MOSC-II-Co molecules.

Another Example includes a device for sensing ions including: a substrate, where the substrate includes metal organic super containers to form a scaffold, and an ion sensing electrode. The device also includes a reference probe. The device also includes where, the device is configured to detect specific ions by measuring electrical response. Implementations according to this Example may include one or more of the following features. The device where the substrate is a mixed matrix membrane. The device where the mixed matrix membrane includes a variety of polymers. The device where the polymer is selected from a group including of polyvinyl chloride, polydimethylsiloxane, poly(methyl methacrylate), urushi, cellulose triacetate, poly aniline, poly urethane, siloprene, poly-(vinyl chloride-co-vinyl acetate-co-hydroxypropyl acrylate), and poly(vinyl chloride-co-vinyl acetate-co-vinyl alcohol). The device where the substrate is a solid sensing surface. The device where the solid sensing surface is selected from a group including of oxide, nitride, and metal. The device further including an ion-sensitive field-effect transistor. The system where the ion sensing electrode is a solid contact electrode and the substrate is placed on the electrode. The system where the system is placed on an ion-sensitive field-effect transistor gate terminal to measure drain current change of the ion-sensitive field-effect transistor. The system where the substrate is incorporated into the ion sensing electrode. The system where the plurality of metal organic super containers are MOSC-II-Co molecules.

Another Example includes a system for sensing molecular ions including a substrate, where the substrate includes a plurality of metal organic super containers designed to selectively bind a target molecular ion, an ion sensing electrode, and a reference electrode, where the target molecular ion is detected by measuring an electrical response. Implementations according to this Example may include one or more of the following features. The system where the ion sensing electrode is a solid contact electrode and the substrate is placed on the electrode. The system where the system is placed on an ion-sensitive field-effect transistor gate terminal to measure drain current change of the ion-sensitive field-effect transistor. The system where the substrate is incorporated into the ion sensing electrode. The system where the plurality of metal organic super containers are MOSC-II-Co molecules.

While multiple implementations are disclosed, still other implementations of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a three-dimensional view of exemplary embodiments of the family of molecules termed MOSCs.

FIG. 2 is a structural representation of a specific MOSC (i.e., MOSC-II-Co) with exemplary MB⁺ binding sites.

FIG. 3 is a depiction of an exemplary embodiment of the nanocavities that provide the tunable structure of the invention.

FIG. 4A is a depiction of an exemplary embodiment of the substrate, a MOSC-MMM.

FIG. 4B is a depiction of an exemplary embodiment of the substrate, a MOSC-SSS.

FIG. 5 is a depiction of an exemplary embodiment of a MOSC-MMM solid-liquid junction sensing interface design.

FIG. 6 is a depiction of an exemplary embodiment of a MOSC-MMM liquid-liquid junction sensing interface design.

FIG. 7 is a depiction of an exemplary embodiment of a MOSC-MMM ISFET sensing interface design.

FIG. 8 is a depiction of an exemplary embodiment of a MOSC-SSS solid-liquid junction sensing interface design.

FIG. 9 is a depiction of an exemplary embodiment of a MOSC-SSS ISFET sensing interface design.

FIG. 10 is a graph of the sensitivity comparison between the MOSC-MMM liquid-liquid junction sensing interface and the MOSC-MMM solid-liquid junction sensing interface, according to exemplary embodiments.

FIG. 11 is a graph of an exemplary embodiment of the impedance measurement of the MMM with and without a MOSC-II-Co.

FIG. 12 is a graph showing an exemplary embodiment of the Nernstian potentiometric response to MB⁺.

FIG. 13 is a graph showing an exemplary embodiment of the sensitivity of MOSC-II-Co MMM ISE to MB⁺, TBA⁺, and K⁺.

FIG. 14 is a graph showing an exemplary embodiment of the response curves of MOSC-II-Co ISE to MB⁺ and TBA⁺.

FIG. 15 is a graph showing an exemplary embodiment of the response time to TBA⁺ molecule, where the time is the time needed to achieve 90% of equilibrium.

FIG. 16 shows an exemplary embodiment of the sensitivity of the MOSC-II-Co MMM ISE for various elemental and small ions.

FIG. 17 is a depiction of an exemplary embodiment of the strong MB⁺ ion capture in a MMM with MOSC content (left) compared to MB⁺ leaching from an MMM without MOSC content (right) and a graph depicting the exemplary absorption of the two samples.

FIG. 18 is a graph showing an exemplary embodiment of the near-Nernstian response of a simulated waste water sample tested using MMM ISE.

FIG. 19A is a depiction of an exemplary embodiment of a silicon nanowire based ion-sensitive field-effect transistor (SiNW-ISFET) covered in electrolyte.

FIG. 19B is a schematic showing the charge separation and equilibrium at the MMM/electrolyte interface, according to an exemplary embodiment.

FIG. 19C is a depiction of an exemplary embodiment of a chip showing an array of SiNW-ISFETs with MB⁺-MMM (left) and Na⁺-MMM (right) formed by drop-casting.

FIG. 19D is a graph showing an exemplary embodiment of I_{DS}-V_{g} transfer characteristics of representative SiNW-ISFETs with and without MMM measured in electrolyte.

FIG. 19E is a graph showing an exemplary embodiment of potential distribution in the SiNW-ISFET with MMM on the gate insulator.

FIG. 20 is a graph showing an exemplary embodiment of ΔV_{TH} of the SiNW-ISFET functionalized with MB⁺-MMM1 as a function of time when α_{MB}_{⁺} was changed from low to high.

FIG. 21 is a graph showing an exemplary embodiment of ΔV_{TH} of the SiNW-ISFET functionalized with MB⁺-MMM1 as a function of α_{MB}_{⁺}, including response to interfering ions such as Na⁺, K⁺, and H⁺.

FIG. 22A is a graph showing an exemplary embodiment of ΔV_{TH} of the SiNW-ISFETs with different MMMs as a function of α_{MB}_{⁺} for SiNW-ISFETs with MB⁺-MMM2 and Control MMM, in three consecutive measurements plotted separately.

FIG. 22B is a graph showing an exemplary embodiment of ΔV_{TH} of the SiNW-ISFETs with different MMMs as a function of α_{MB}_{⁺} in response curves averaged from three measurements.

FIG. 23A is a graph showing an exemplary embodiment of ΔV_{TH} of the SiNW-ISFETs functionalized with Na⁺-MMM as a function of time when α_{Na}_{⁺} was changed from low to high.

FIG. 23B is a graph showing an exemplary embodiment of ΔV_{TH} of the SiNW-ISFETs functionalized with Na⁺-MMM as a function of α_{Na}_{⁺}, α_{K}_{⁺}, and α_{MB}_{⁺}.

FIG. 24 is a graph showing an exemplary embodiment of ΔV_{TH} of the SiNW-ISFETs functionalized with MB⁺-MMM1 and Na⁺-MMM as a function of α_{MB}_{⁺} and α_{Na}_{⁺}, respectively, with a concentration series prepared using DI water and river water.

FIG. 25 is a graph showing an exemplary embodiment of multiplexed measurement of MB⁺ and Na⁺ in one solution with the concentration series prepared with DI water (solid line) and river water (dash line).

### DETAILED DESCRIPTION

The various embodiments disclosed or contemplated herein relate to the use of metal-organic supercontainers (MOSCs) as a size-selective ionophore by incorporation into a substrate such as a mixed-matrix membrane (MMM) for ion-selective electrodes (ISEs).

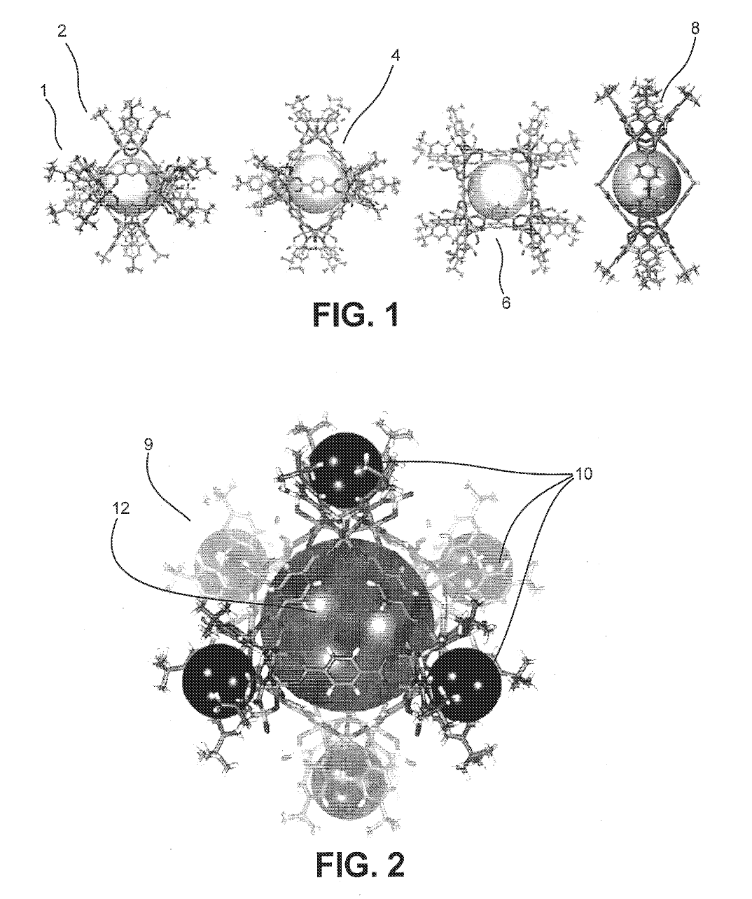

The discovery of a new family of coordination container molecules—metal-organic supercontainers (MOSCs) shown in FIG. 1—resulted in structurally unique and diverse container molecules with a structurally unique, multi-pore architecture that displays both endo- and exo- nano-cavities with chemically tunable structures. It is understood that the exo- and endo- cavities mimic the binding pockets of proteins and enzymes.

As shown in FIG. 1, the MOSC structures may include, but are not limited to, face-directed octahedral 2, edge-directed octahedral 4, barrel-shaped 6, or cylinder-shaped 8 geometries using trigonal, linear, angular-planar, and angular-nonplanar carboxylate linkers, respectively, linking metal ions and sulfonylcalix[4]arenes. It is understood that further structures are possible. Additional discussion is found in U.S. patent application Ser. No. 13/862,651 which is incorporated by reference in its entirety for all purposes.

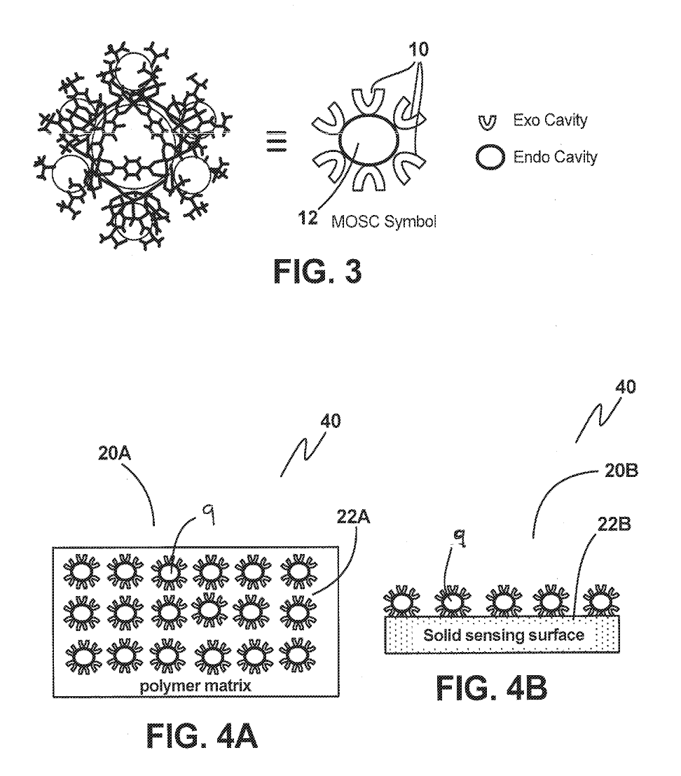

As best shown in FIG. 2, and simplified in FIG. 3, one aspect that makes MOSCs structurally unique is a multi-pore architecture that displays both exo-cavities 10 and endo-cavities 12. In exemplary implementations the cavities are chemically tunable structures that give the MOSC molecules the ability to function as extremely efficient host systems. This structural feature is important because the ability to design multiple nanocavities within a single host molecule capable of selecting ion species in a tunable fashion makes MOSCs extremely desirable for ion-specific sensing devices. The exemplary embodiment shown in FIGS. 2 and 3, is the MOSC-II-Co molecule 9 that selectively binds methylene blue (MB⁺), for example one MB⁺ per cavity, with an apparent binding constant of 1.42×10⁴ M⁻¹. It is understood that further implementations are possible.

As a result of the presence of these hierarchical cavities and their chemically tunable structure, MOSCs function as extremely efficient host systems. MOSCs are unique in their ability to show multiple nanocavities within a single host molecule, making them capable of selectively recognizing ionic species in a tunable fashion and giving them an advantage in generating ion-specific sensing devices. Particularly, the MOSC's tunable structure and solution processability make them very attractive for selective ion sensing for molecular ions. The presently disclosed system relates the use of MOSCs as a new type of ionophore at a sensor/sample interface in a selective ion detection system. MOSCs are described in U.S. patent application Ser. No. 13/862,651 which was incorporated by reference in its entirety for all purposes.

As best shown in FIGS. 4A-B, in exemplary implementations of the system 40 the MOSCs are incorporated into a substrate to form a scaffold 20A, 20B. In the examples of FIGS. 4A-B, the MOSC-substrate scaffolds 20A, 20B can take several forms, two non-limiting exemplary implementations being an MOSC-MMM 20A, comprising MOSC molecules 9 and a polymer matrix 22A (FIG. 4A), or an MOSC-solid sensing surface (SSS) scaffold 20B, comprising MOSC molecules 9 and an SSS substrate 22B (FIG. 4B).

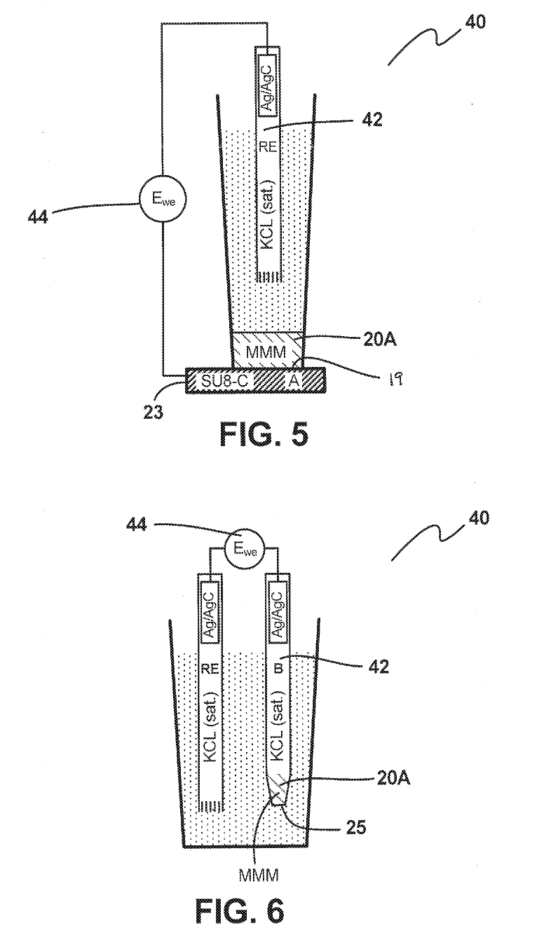

As shown in FIGS. 4A-B and FIGS. 5-7, in various implementations, the MOSC 9 is directly incorporated into a MMM 22A to form the scaffold 20A. In these and other implementations, the MMM 22A may contain a variety of polymers. It is understood that other matrices, including, but not limited to, polydimethylsiloxane, poly(methyl methacrylate), Urushi, cellulose triacetate, poly aniline, poly urethane, siloprene, Poly-(vinyl chloride-co-vinyl acetate-co-hydroxypropyl acrylate), and Poly(vinyl chloride-co-vinyl acetate-co-vinyl alcohol) can also be utilized to form the MMM 22A.

In these implementations, the ion sensing device or system 40 therefore has a MOSC-MMM 20A structure, in which the MOSC molecules are directly incorporated into a polyvinyl chloride (PVC) based MMM. For example, the device might be placed on a solid contact (SC) and the electrical response between the solid-liquid sensing interface could be measured. Alternatively, the device or system 40 might be used to measure the electrical response between liquid-liquid junction sensing interfaces. In another example, the device might be used in conjunction with an ion-sensitive field-effect transistor 62 (ISFET) by placing the device on the gate terminal 21 of the ISFET 62 and measuring the drain current change of the ISFET 62.

Accordingly, discussed herein are various implementations relating to a device, system and methods for the use of MOSCs as a size-selective ionophore by incorporation into a substrate such as an MMM for ISEs. While there are many commercially available ionophores, the devices and methods currently available are useful mainly for detection of elemental or other small inorganic ions. The disclosed systems, methods, and devices allow for rapid and precise monitoring of molecular (e.g., organic) ions for environmental and biomedical applications.

In certain implementations, the device or system comprises a MOSC-solid sensing surface (SSS) structure 20B, in which chemical bonds allow the MOSCs to bind onto an SSS 20B, such as an oxide, nitride, or metal. For example, the MOSC-SSS 20B could be used to measure the electrical response between a solid-liquid junction sensing interface. Alternately, the device might be used in conjunction with an ISFET 62 by placing the device on the gate terminal 21 of the ISFET 62 and measuring the drain current change of the ISFET 62.

In various implementations, the size of the binding cavity and the number of binding sites (as shown for example in FIGS. 1-3), is created by altering constituent elements of the MOSCs. These cavities can be tuned to enable the design of a device for detection of a highly specific ion. For example, in Example 1 (discussed below) this implementation is demonstrated using methylene blue (MB⁺). It is understood that further implementations are of course possible.

Additionally, Example 1 shows this implementation using Tetrabutylammonium (TBA⁺). In various implementations the device has a decreased response to elemental and other small inorganic ions, providing an extremely effective measurement for local concentration of the larger target molecular ion. Other implementations are possible.

In use, and as best shown in FIG. 5, in certain implementations, the MMM scaffold 20A is placed on an solid contact (SC) electrode 23 and used in conjunction with a reference electrode 42 to create a system 40. The system 40 of these implementations is used to measure the electrical response (shown at 44) between the system's 40 solid-liquid junction sensing interface (shown generally at 19).

For example, to make an SC electrode 23 the MOSC might be incorporated by spin-coating a 1.5 μm SU8 layer on top of an SiO₂-covered Si wafer with an oxide thickness of 650 nm. In this implementation, the SU8 film can then be pyrolyzed in a quartz tube flow-through furnace at 900 degrees Celsius in the reducing atmosphere of 95 percent N₂ and 5 percent H₂ for one hour. Then polyethylene wells with inner diameters of about 6 mm can be glued on top of the SU80-derived carbon using a quick setting epoxy. Finally, 40 μL of MMM solution is drop-casted inside the fabricated well on top of the pyrolyzed SU8, making a thin-film coating over the entire carbon electrode.

Turning to the implementation of FIG. 6, in this embodiment the system 40 includes the scaffold 20A and the reference electrode 42 is used to measure the electrical response (designated at 44) between the liquid-liquid interface. In various implementations, the reference electrode 42 can be an Ag/AgCl reference electrode or another reference electrode known to those with skill in the art.

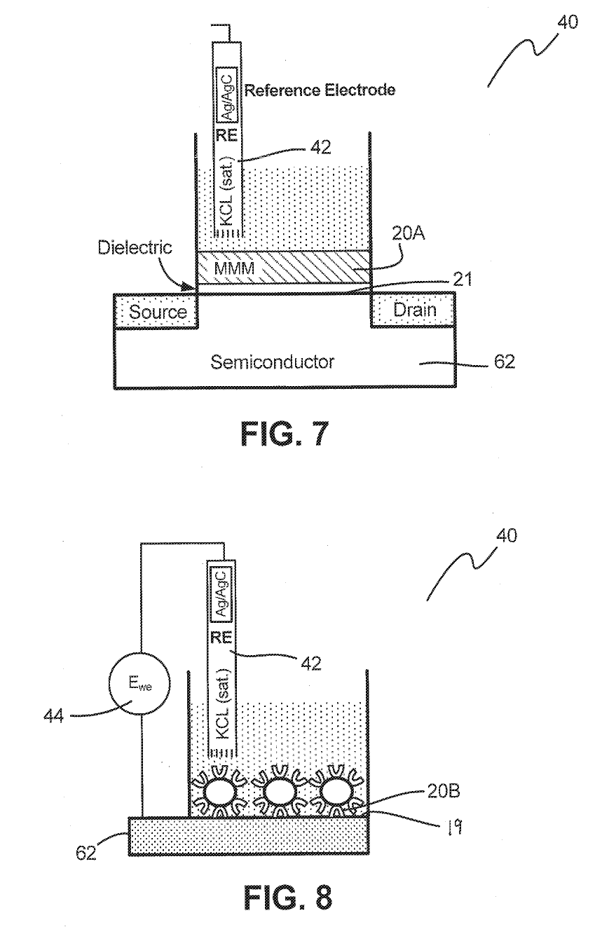

As best shown in FIG. 7, in certain implementations the system 40 includes the scaffold 20A and the reference electrode 42. The system 40 is placed on the gate terminal 21 of an ISFET 62 and used to measure the drain current change of the ISFET 62, as would be understood.

As shown in FIGS. 4 and 7-9, in various implementations the MOSC 9 is incorporated into an SSS 22B to form a scaffold 20B. In certain implementations, the SSS 22B can be oxide, nitride, metal or other solid sensing surface structures, as would be understood. In certain implementations, the MOSC 9 can be attached to the SSS 22B with chemical bonds, weak non-covalent interactions, covalent bonds such as S—Au (i.e., sulfur-gold) linkages, or other means of attachment known to those with skill in the art.

Similar to the MMM scaffold 20A depicted in the implementation of FIG. 5, in certain implementations, as shown in FIG. 8, a SSS scaffold 20B can be used in conjunction with a reference electrode 42 to form the system 40, which can be used to measure the electrical response 44 between a solid-liquid interface, as would be understood. Many alternate implementations are possible.

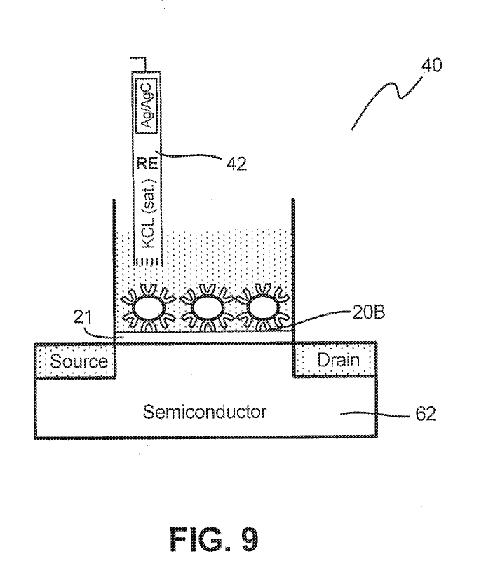

As shown in FIG. 9, in certain alternate implementations, the scaffold 20B and the reference electrode 42 will form the system 40 that can be placed on the gate terminal 21 of an ISFET 62. In these and other implementations, the system 40 can thereby be used to measure the relative or absolute drain current change of the ISFET.

In exemplary implementations, and as discussed in the examples below, the system 40 demonstrates a near-Nernstian response, a reduction in charge-transfer resistance (both shown in Example 1), and little to no visible leeching (shown in Example 2), indicating that the system 40 is highly effective in rapid and precise monitoring of molecular ions. In all implementations, the system 40 is environmentally stable (shown in Example 3). In various implementations, the system can include an ISFET for monitoring molecular ions with little interference from elemental or other small inorganic ions (shown in Example 4).

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

An exemplary study demonstrated herein in Examples 1-3 is found in “Biomimetic supercontainers for size-selective electrochemical sensing of molecular ions” by Netzer, N. L., Must, I., Qiao, Y., Zhang, S., Wang, Z., Zhang, Z. (Sci. Rep, 2017, 7, 45786), and is hereby incorporated by reference in its entirety for all purposes.

### EXAMPLE 1: PROOF-OF-CONCEPT STUDY WITH MB⁺ AND TBA⁺

The present example investigates the ability of a specialized MOSC, designated as MOSC-II-Co 9 (shown in FIG. 2), to selectively bind MB⁺ in both solution and in solid-state. The MOSC-II-Co molecule 9 was designed with its cavities matching the size of MB⁺, a known water contaminant. Both the MOSC and MB⁺ were combined with a polymeric MMM and incorporated into an MB⁺-selective electrode in the nanomolar to micromolar range. The results demonstrated the encouraging potential of MOSC-based ion sensors via exploiting the tunable nature and specific molecular recognition capability of the MOSC structures.

### Materials and Methods

The exemplary MOSC-II-Co 9 was obtained from the reaction of Co(II), p-tert-butylsulfonyl-calix[4]arene, and 1,4-benzedicarboxylate. It had an edge-directed octahedral geometry and features an outer diameter of 3.3 nm, an inner diameter of 1.7 nm, and an internal volume of 1.2 nm³. It possesses a total of seven well-defined binding domains, including six exo-cavities (measuring 0.74 nm) and one endo-cavity (measuring 1.7 nm). The size of the cavities were designed to fit the dimensions of MB⁺, which has a length of 1.6 nm and a width of 0.7 nm.

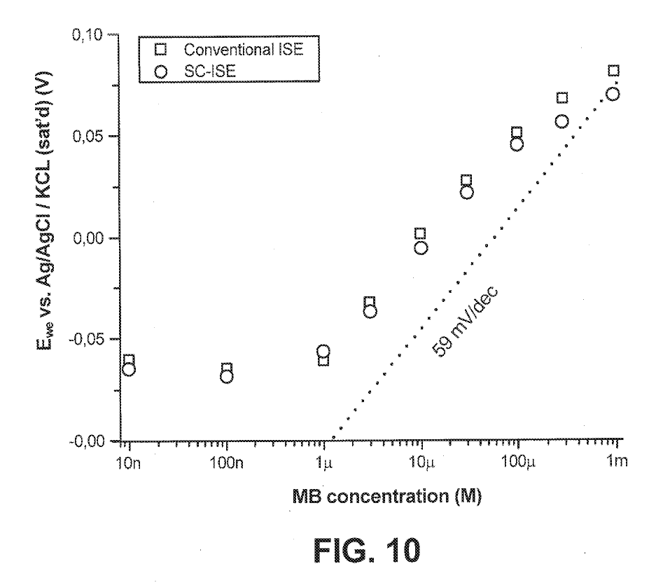

After the MOSC-II-Co molecule 9 was created, it was combined with an MMM and incorporated onto an electrode to form an ion sensing device. For the study, a conventional ISE with an inner filling solution was chosen because of its versatility and ease of set-up. To facilitate reversible sorption of MB⁺ and fabricate a working MB⁺-ion-selective MMM using MOSC-II-Co, the ionic site of tetrakis(4-chlorophenyl)borate, MB⁺, and MOSC-II-Co 9 were dispersed into a solution of tetrahydrofuran (THF) and PVC. After the MMM solution was allowed to mix for two hours, an ISE with an inner filling solution and an internal Ag/AgCl reference was manufactured. First, the MOSC-II-Co MMM solution was drawn up into an empty pipette tip via capillary force. Once the membrane was set and the MOSC-II-Co MMM was formed at the tip of the tube, the inner filling solution and Ag/AgCl pellet were added. The membrane thickness was about 0.5 nm. The MOSC-II-Co MMM ISE's potential (E_{we}) 44 was recorded versus a standard Ag/AgCl reference electrode, shown in FIG. 10. A solid-contact ISE was also fabricated with a pyrolyzed SU8-derived carbon (SU8-C) electrode. A simple drop-cast method was used to apply the MOSC-II-Co MMM onto the SU8-C electrode to create the MOSC-II-Co MMM-SC ISE. The MOSC-II-Co MMM-SC ISE was also recorded versus Ag/AgCl reference electrode, shown in FIG. 10. As FIG. 10 shows, there is little variation between the two methods.

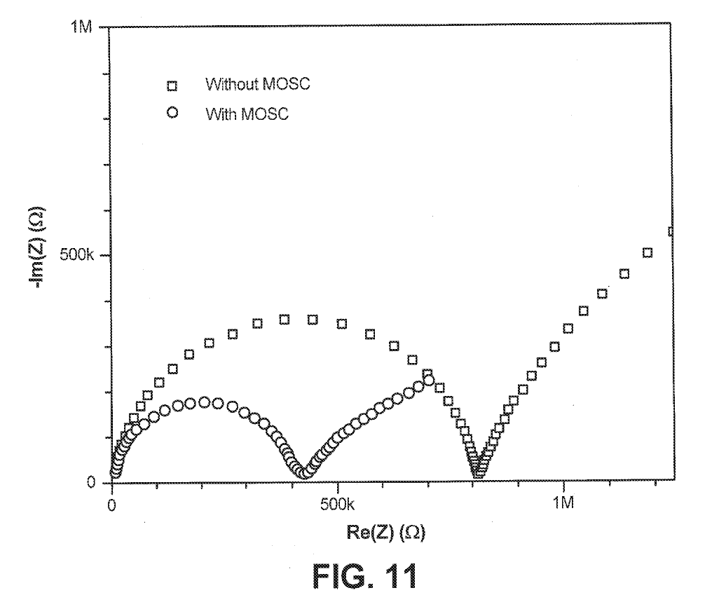

The characteristics of the MOSC-II-Co MMM ISEs were investigated using both impedance spectroscopy and potentiometry. As shown in FIG. 11, the impedance of the MMM with MOSC-II-Co 9 reveals a reduction of the charge-transfer resistance by a factor of two when compared to the MMM without MOSC-II-Co 9. This is a strong indication of MOSC-II-Co 9 promoting MB⁺ ion transfer across the MMM-analyte interface, which shows that the MOSC molecule is a good candidate for potentiometric sensing. The MOSC-II-Co MMM ISEs were also investigated using K⁺, Ag⁺, and NH₄⁺ to test binding of small molecules, and with TBA⁺ to test the binding of larger molecules with a similar structure to that of MB⁺.

### Results

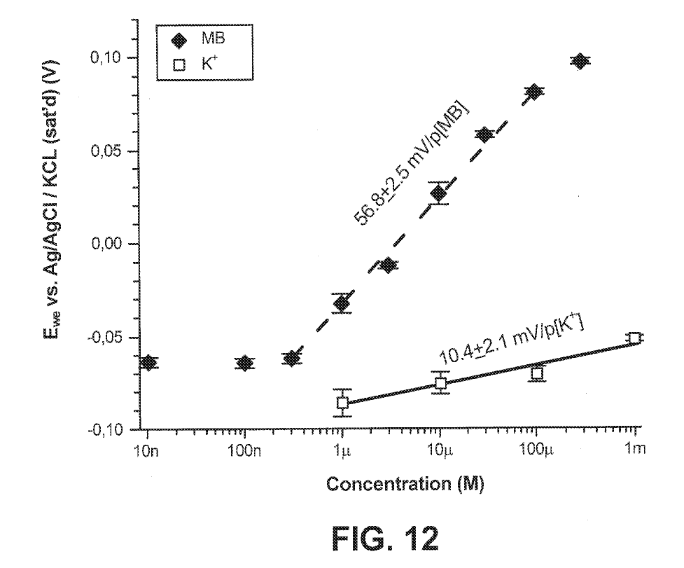

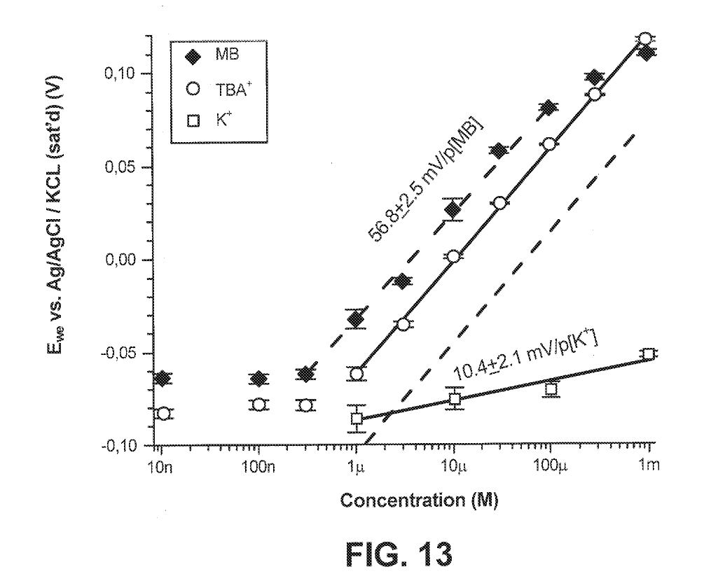

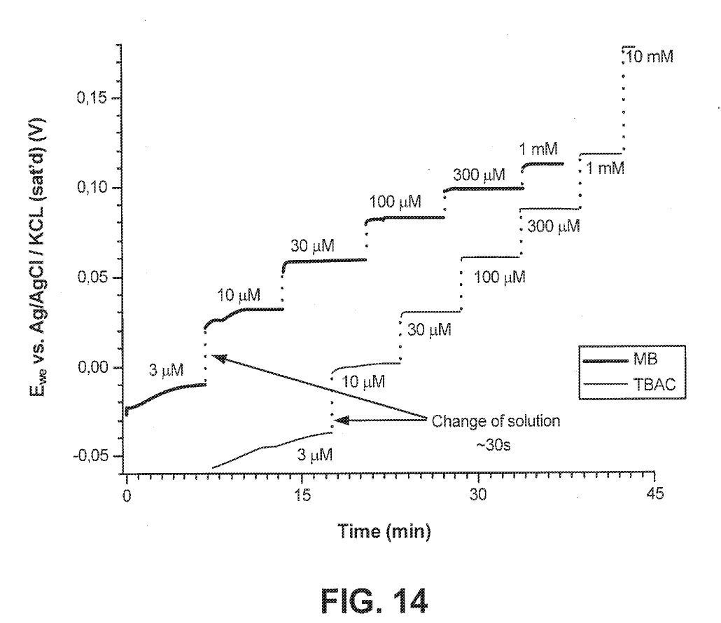

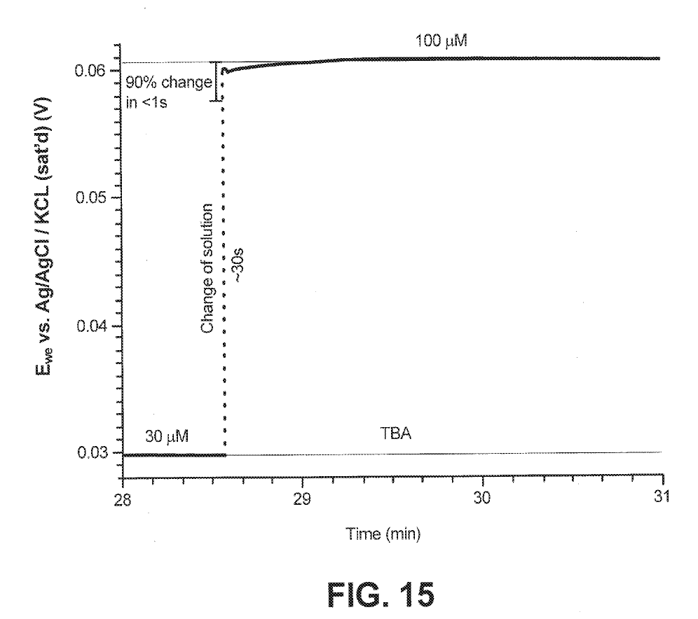

The potentiometric response shown in FIG. 12 is evidence of the ideal match of MB⁺'s dimensions with those of the MOSC cavities. The MOSC-II-Co MMM ISE gives a near-Nernstian response to the MB⁺ concentration, providing clear evidence for the suitability of MOSC-II-Co 9 as an ionophore and its direct implementation in ISE sensors. The size-selectivity was further verified by the response showing the binding event for TBA⁺, depicted in FIGS. 13 and 14. As shown in FIG. 15, the response time for the binding event was also extremely short and the potential stability was extremely high for the TBA⁺ test, further indicating the suitability of MOSC-II-Co 9 as an ionophore.

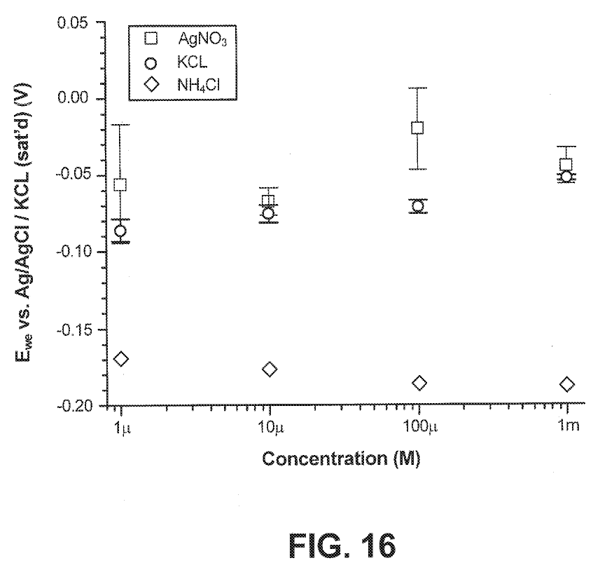

As also shown in FIGS. 13 and 16, the device expressed a very minimal response to the smaller ions, likely because their sizes were too small to allow effective binding. This is likely because the lower rim of the MOSC-II-Co 9 precursor calixarene serves only as a structural site by coordinating to Co(II) using its phenolic oxygen atoms. This simultaneously increases formation of critical MOSC structures while decreasing affinity to small metal ions because of a lack of binding groups. In addition, the six exo-cavities are orderly pre-organized around the central endo-cavity, providing an effective strategy to promote local concentration of the target ion while decreasing sensitivity to small metal ions.

### EXAMPLE 2: LEECHING STUDY

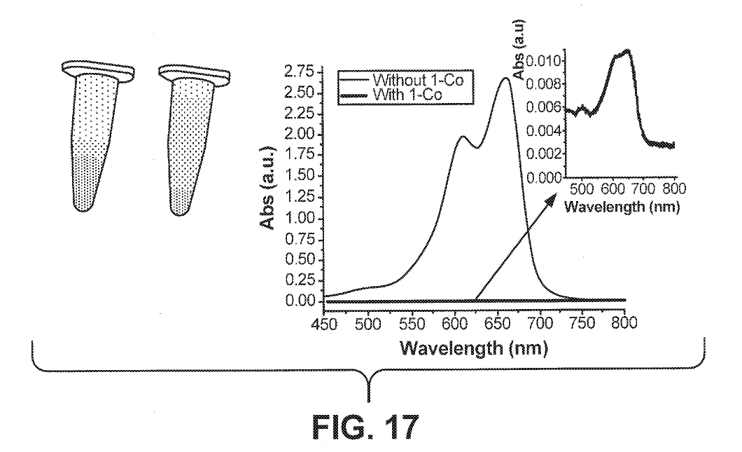

A simple leeching test was performed by preparing two MMMs, one with MOSC-II-Co 9 and one without. All other constituents were kept constant. The MMMs were stored in deionized (DI) water for over one week. As shown in FIG. 17, no change in the color of the water for the MOSC-MMM was observed, indicating that MB⁺ was captured by MOSC-II-Co 9. However, the solution in contact with the MMM without MOSC-II-Co turned dark blue, indicating that the MB⁺ had leached out. Additionally, the ISEs used for measurement showed no dependence on MB⁺ concentration, strongly indicating that the MB⁺ sensitivity results from the inclusion of the MOSC.

### EXAMPLE 3: SIMULATED WASTE WATER SAMPLE

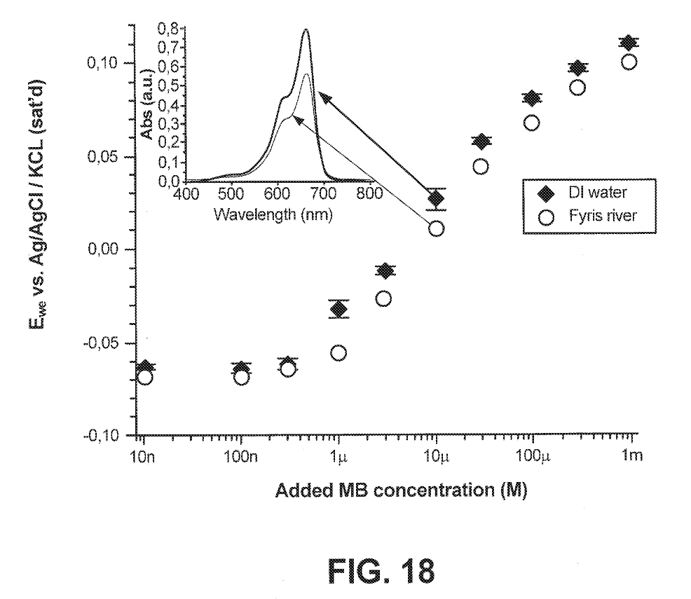

The MOSC-II-Co molecule 9 was tested in a simulated wastewater sample collected from the Fyris River in the city of Uppsala, Sweden to illustrate that the proof of concept protocols can be applied in a more practical setting. A wastewater sample was collected and controlled amounts of the pollutant MB⁺ were added. As demonstrated in FIG. 18, the response curve for the simulated waste-water remained near-Nernstian. Although FIG. 18 shows an apparent shift in the detection limit towards higher concentrations of MB⁺, the shift can be attributed to decreased oxygen levels in the sample caused by microorganism found in the river water, verified by the control experiment, in which an equal amount of MB⁺ was dissolved in DI water and water from the Fyris River. The loss of oxygen produces a reducing environment, which can in turn decrease the actual MB⁺ ion concentration by a certain amount and cause a small shift in the response curve.

### EXAMPLE 4: MOLECULAR ION SENSING

This example demonstrates uses of silicon nanowire based ISFET 62 (SiNW-ISFET) for electronic sensing of ions in conjunction with MOSC-incorporated MMMs 20A, as is shown generally in FIG. 7 for example.

### Materials and Methods

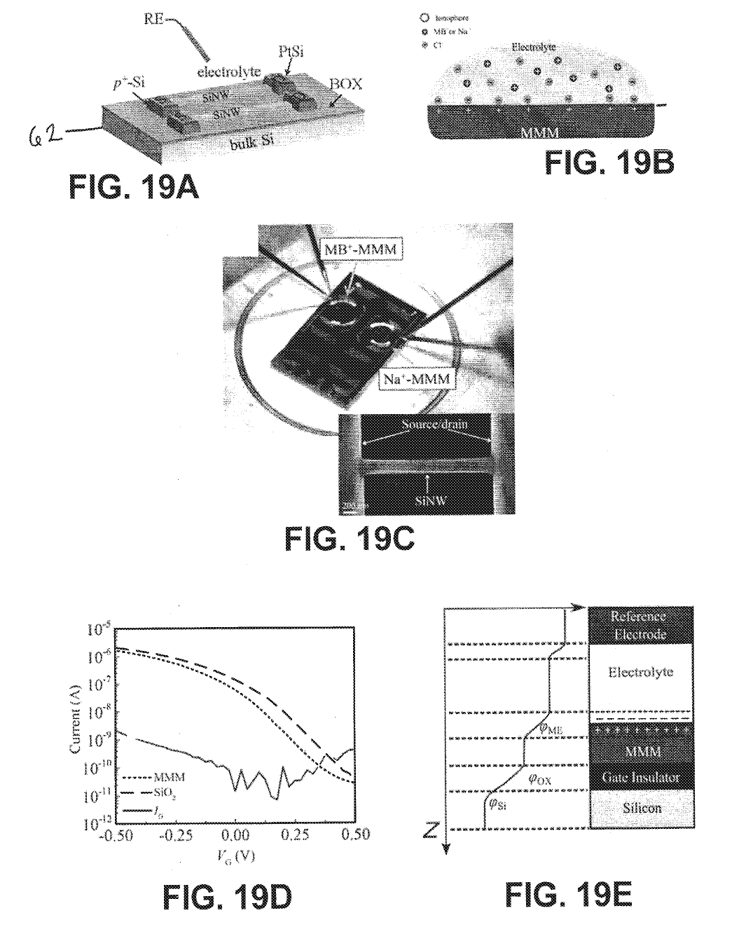

The SiNW-ISFET chips were fabricated using standard silicon process technology on silicon-on-insulator wafers. The silicon layer in the channel region was thinned down from 260 to 40 nm via thermal oxidation. SiNWs were first defined by lithography and dry etching, and then were laterally shrunk to the desired width. To reduce the series resistance, PtSi/p⁺-Si leads were used for connecting the SiNW-ISFETs 62 to the contact pads placed at the edges of the chip. Finally, a fresh thin silicon oxide (SiO₂) film was grown via rapid thermal oxidation to serve as gate insulator and passivation on the chip. An exemplary embodiment of SiNW-ISFETs covered by electrolyte are shown in FIG. 19A.

Four types of MMMs were prepared: MOSC-doped MMM premixed with MB (MB⁺-MMM1), MMM premixed with MB⁺ but without MOSC (MB⁺-MMM2), Na-ionophore doped MMM (Na⁺-MMM), and blank control MMM containing only ionic sites (Control-MMM). Once the solution was prepared and there were no visible particles, the MMMs were fabricated by drop casting the solution on the device area of the chips by pipettes. An exemplary embodiment of a chip formed by drop-casting is show in FIG. 19C. Then, the chips with MB⁺-MMM1 and MB⁺-MMM2 were placed in a fume hood for approximately 2 hours while the chips with Na⁺-MMM were left in ordinary atmosphere overnight. Before any measurement was performed, the chips with MB⁺-MMM1 and MB⁺-MMM2 were conditioned in a 10 μM MB⁺ solution overnight while the chips with Na⁺-MMM were conditioned in a 100 mM NaCl solution for 4 hours. All procedures of MMM preparation and conditioning were conducted at room temperature.

All electrical measurements were performed at room temperature on a probe-station using a Keysight B1500A precision semiconductor parameter analyzer. To facilitate measurement with electrolyte, a polydimethylsiloxane (PDMS) container was placed on the chip. During the measurement, the gate potential (V_{G}) was kept constant and was applied to an Ag/AgCl reference electrode 42 (with 3.4 M KCl as filling electrolyte, purchased from Harvard Apparatus) immersed in the solution. The SiNW-ISFET 62 was biased in its subthreshold region. The drain-to-source current (I_{Ds}) was monitored in real-time with a constant V_{DS}=1 V. Solution exchanges during the measurement were realized manually using a pipette. In detail, each measurement was initiated with a solution with a low sample concentration in the PDMS container in order to set an I_{DS} baseline. Once the baseline became stable, the concentration in the container was increased by adding samples of higher analyte concentrations. Similar solution-exchange procedures were applied to the multiplexed detection, using a starting solution containing both molecular and elemental ions of low concentrations.

### Results

Characterization of SiNW-ISFETs. The transfer characteristics, i.e., I_{DS} vs. V_{G}, of two SiNW-ISFETs 62 with and without MMM measured in the same electrolyte are shown in FIG. 19D. The two SiNW-ISFETs 62 have the same channel dimensions, i.e., 2 μm in length, 200 nm in width, and 40 nm in height. Both devices exhibit similar subthreshold slope (SS), i.e., 118 mV/dec, while surface functionalization with MMM 20A has clearly decreased the threshold voltage (V_{TH}) from that of the device with bare gate insulator. As shown in the potential diagram in FIG. 19E, when a reference electrode is used, the electrical potential of the electrolyte (ψ_{EL}) is fixed by V_{G}. For the SiO₂/electrolyte interface, its potential (φ_{OE}) is determined by the pH value of the electrolyte since the reaction between H⁺ in the electrolyte and the silanol groups (Si—OH) on the SiO₂ surface is mainly responsible for surface charging. However, when the gate insulator is covered by MMM 20A, the potential at the MMM/electrolyte interface 19 (φ_{ME}) is governed by the binding affinity between the ionophores and target ions, as well as the ionophores and ion concentrations in the MMM 20A and the electrolyte, respectively. Given the same type of MMM 20A and same ionic composition in the electrolyte, φ_{ME} should remain unaltered. The observed difference in V_{TH} for the two SiNW-ISFETs 62 is, thus, the consequence of φ_{ME} departing from φ_{OE}. The negligible change of SS by MMM 20A is essential for maintaining the gate coupling efficiency and current sensitivity of the SiNW-ISFET 62. This is due to the significantly reduced resistivity of MMM 20A after the addition of ionic sites, leading to negligible potential drop across the MMM 20A bulk when it is in series connection with the gate insulator as illustrated in FIG. 19E. It is worth noting that the gate leakage (I_{G}) is below 2 nA for V_{G} ranging from −0.5 to 0.5 V, which indicates negligible parasitic current in the system during the measurement and is important for a stable sensor operation in the electrolyte.

Molecular Ion Sensing. The detection of MB⁺ using the MB⁺-MMM1 20A functionalized SiNW-ISFET 62 relies on the size-selective feature of the interaction between the MOSC molecules and the MB⁺ ions. The MOSC possesses one endo- (Ø˜1.7 nm) and six exo-cavities (Ø˜0.74 nm), which determines its ion-capture properties. The sizes of these cavities fit with the dimensions of MB⁺ that measure 1.6 nm in length and 0.7 nm in width. It has been shown that, the MOSC-II-CO 9 has the ability to selectively bind to MB⁺ in both solution and solid-state with an apparent binding constant of (1.42±0.31)×10⁴ M⁻¹. This favorable binding is believed to be due in part to the so-called “cation-π” interaction between MB⁺ and the MOSC cavity, which feature a positive charge and multiple aromatic groups (aka π-systems), respectively.

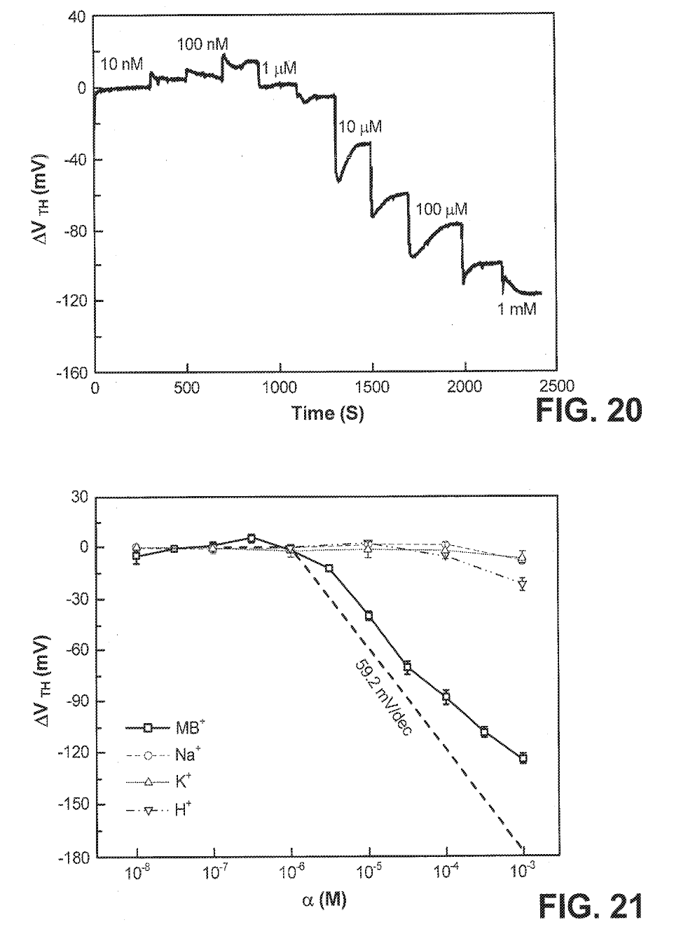

FIG. 20 shows ΔV_{TH} of the MB⁺-MMM 20A functionalized SiNW-ISFET 62 as a function of time when MB⁺ activity, i.e., α_{MB}_{⁺}, is increased from 10 nM to 1 mM. All V_{TH} shifts are retrieved with respect to the V_{TH} value at α_{MB}_{⁺}=10 nM, i.e., ΔV_{TH}=0 mV at α_{MB}_{⁺}=10 nM. As α_{MB}_{⁺} increases, the MMM 20A surface becomes more positively charged (more negatively charged on the electrolyte side), leading to a negative shift of V_{TH} and lowering of I_{DS} for the SiNW-ISFET 62 with hole conduction. The overshoot after each addition of sample solution could be related to the way of sample mixing in the PDMS container, i.e., each increment of sample concentration is performed by adding a sample with higher concentration to mix with the sample already in the container. Although gently performed with the sample addition, the transport of ions and molecules in the electrolyte is governed by convection instead of diffusion. This explains the observed instantaneous response of V_{TH} to sample addition in FIG. 20, and most likely the overshoot as well. A slight positive shift of V_{TH} with increasing α_{MB}_{⁺} is visible when α_{MB}_{⁺} is lower than 1 μM. This could have arisen from residues of previous measurements at higher α_{MB}_{⁺}. As a result, the activities at the extremely low end could be higher than anticipated.

The variation of V_{TH} with α_{MB}_{⁺} is depicted in FIG. 21, with each data point representing an average of three independent measurements. The MB⁺-MMM1 20A functionalized SiNW-ISFET 62 shows a near-Nernstian response to α_{MB}_{⁺} with a slope of 56.4±1.8 mV/dec up to α_{MB}_{⁺}=30 μM. The change of V_{TH} starts to deviate from the ideal trend, giving rise to a slope of 35.8±1.4 mV/dec when α_{MB}_{⁺} is above 100 μM. Such a deviation at high α_{MB}_{⁺} can be explained by the co-extraction of MB⁺ and Cl⁻ from the sample into MMM 20A, leading to the so-called Donnan failure. This can be mitigated by further optimization of the MMM 20A composition, e.g., ratio of ionophore to ionic site. The lower detection limit extrapolated from the MB⁺ response curve is ˜1 μM. The performance of the SiNW-ISFET 62 based MB⁺ sensor is close to MOSC-incorporated conventional ISE, and is also comparable with MB⁺ ISEs with different ion receptors and membrane compositions. The result shows a successful integration of MOSC-doped MMM 20A with SiNW-ISFET 62 as well as its excellent repeatability in potentiometric MB⁺ sensing.

The MB⁺-MMM1 20A functionalized SiNW-ISFET 62 was further investigated for its response to common interfering elemental ions. As shown in FIG. 21, no substantial shift in V_{TH} of the SiNW-ISFET 62 is observed with α_{Na}_{⁺}, α_{K}_{⁺}, and α_{H}_{⁺} up to 100 μM, presumably because these ions are too small in size to allow for effective competition with the MOSC cavities. When the ion activities were further increased from 100 μM to 10 mM, the SiNW-ISFET 62 started to respond, giving rise to a slope of 13.2, 8.71, and 13.5 mV/dec for Na⁺, K⁺, and H⁺, respectively. Similar responses at high ion activities (except for H⁺) were also observed for the SiNW-ISFET 62 without MMM 20A, i.e., with bare SiO₂, indicating that such responses are more likely due to changes in ionic strength and are not related to the binding between the MOSC molecules and the ions. The superior selectivity against elemental ions is a clear advantage of using MOSCs for recognition of molecular ions in the physiological and environmental processes.

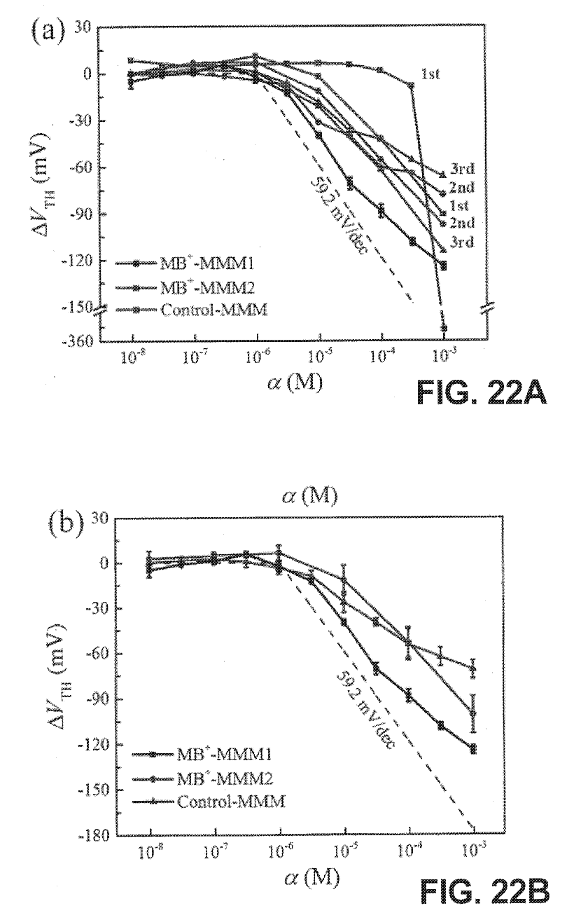

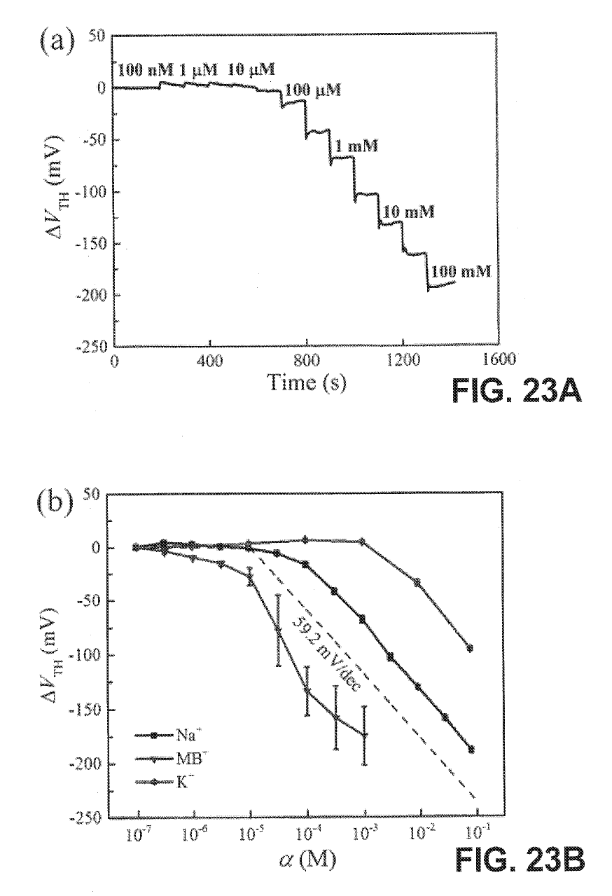

To achieve the desired Nernstian response, it is crucial to maintain a constant activity of the ion of interest in the bulk of the membrane phase. Without MOSC molecules incorporated into the MMM 20A, MB⁺ will gradually leach into the aqueous phase, leading to a drift of α_{MB}_{⁺} in the membrane phase. Three consecutive response curves of the SiNW-ISFET 62 functionalized with such MMM 20A without MOSC incorporation, i.e., MB⁺-MMM2 20A, are depicted in FIG. 22A-B. The response curve of the MB⁺-MMM1 20A functionalized SiNW-ISFET 62 is also included as a reference. Due to the instability of α_{MB}_{⁺} in the membrane phase, the sensor response deviates significantly from the ideal Nernstian behavior. Moreover, the reproducibility is poor in comparison with the reference, as evident by its substantially larger standard deviation shown in FIG. 22B. The lower detection limit of the MB⁺-MMM2 20A functionalized SiNW-ISFET 62 is also inferior to the reference, which could be explained by MB⁺ leaching out from MB⁺-MMM2 20A, thereby considerably raising α_{MB}_{⁺} at the interface, i.e., α_{MB}_{⁺}^{IF}. In another case where MMM 20A contained neither premixed ion of interest (MB⁺) nor MOSC molecules, designated as Control-MMM 20A, the sensor showed negligible potentiometric response during the first measurement. This is expected since there is no MB⁺ in the MMM 20A to balance the charge and to establish a stable phase boundary potential with the MB⁺ in the solution. However, MB⁺ is relatively hydrophobic. As the SiNW-ISFET 62 with Control-MMM 20A is used over and over again, the hydrophobic MB⁺ can become incorporated into the membrane and thereby rendering it an ion-exchange membrane. By repeating the measurement for the second and third time, α_{MB}_{⁺} in Control-MMM 20A increases and the SiNW-ISFET 62 sensor starts to respond with a lower detection limit similar to the reference. However, the response curve averaged from three measurements, shown in FIG. 23A, significantly deviates from the ideal Nernstian behavior. Such a sub-Nernstian response could be ascribed to a sample dependent α_{MB}_{⁺} in Control-MMM 20A, i.e., α_{MB}_{⁺} is not constant in the membrane phase but is dependent on the MB⁺ activity in the aqueous phase. The results here clearly demonstrate the critical role of the MOSC molecules for stabilization of the activity of the ion of interest in MMM 20A, which is essential for achieving a Nernstian response.

Elemental Ion Sensing. FIG. 23A shows ΔV_{TH} of a Na⁺-MMM functionalized SiNW-ISFET 62 as a function of time with increasing α_{Na}_{⁺}. An overshot is also visible after addition of each new sample solution but the recovery is significantly faster than observed in MB⁺ sensing, which could be explained by the relatively higher diffusivity of Na⁺ comparing to the bulkier MB⁺ in the solution and thus shorter time for the solution to become homogenized. As depicted in FIG. 23B, in the presence of K⁺ as a likely interfering ion with α_{K}_{⁺}=1 mM, the Na⁺-MMM 20A functionalized SiNW-ISFET 62 exhibits a near-Nernstian response with a slope of 57.9±0.7 mV/dec in a wide α_{Na}_{⁺} range from 100 μM to 100 mM, with a lower detection limit of ˜60 μm. Such a performance is in close match with the reported data obtained from conventional ISE with the same Na-ionophore. On the other hand, the response to K⁺, in the presence of Na⁺ as a likely interfering ion with α_{Na}_{⁺}=1 mM, becomes close to Nernstian (˜61 mV/dec) at much higher concentrations, i.e., α_{K}_{⁺}>10 mM. A selectivity coefficient K_{Na, K}^{Pot} of 10^{-1.2} can be estimated from the lower detection limit of Na⁺ (α_{Na}_{⁺}(DL)) in the presence of interfering K⁺ with an activity of α_{K}_{⁺} through:

α_{Na}_{⁺}(DL))=K_{Na, K}^{Pot}α_{K}_{⁺}, (Equation 1)

which is in good agreement with the reported values.

Considering the abundance of molecular ions in physiological samples, it is important to investigate the cross-sensitivity of the elemental ion sensor towards molecular ions. The response curve of the Na⁺-MMM 20A functionalized SiNW-ISFET 62 towards the molecular ion MB⁺ is shown in FIG. 23B. Similar to the case of Control-MMM 20A, the hydrophobic MB⁺ is incorporated into the Na⁺-MMM 20A during measurement. The SiNW-ISFET 62 exhibits a super-Nernstian response towards MB⁺ once α_{MB}_{⁺} is above a critical threshold, i.e., ˜10 μM in this experiment, with a large standard deviation among the measurements. Therefore, in order to have a controllable detection of elemental ions, it is essential to keep the activity of hydrophobic ions below the threshold where they start to be incorporated into MMM 20A.

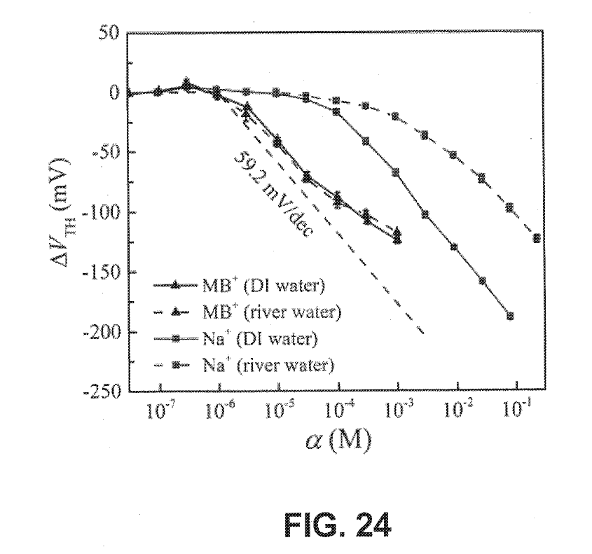

Detections with River Water. To prove the herein disclosed sensors can be utilized for complex sample, both molecular and elemental ion detection experiments were performed with the water collected from the Fyris River in the city of Uppsala, Sweden. The response curves of the MB⁺-MMM1 20A functionalized SiNW-ISFET 62 are undistinguishable between the river water and the DI water depicted in FIG. 24. These results clearly demonstrate the robustness of the SiNW-ISFET 62 based molecular ion sensor, which can be attributed to its remarkable selectivity against interfering elemental ions often abundantly present in river water. Similar experiments were conducted with the Na⁺-MMM 20A functionalized SiNW-ISFET 62. A significant performance deterioration is observed as evident by a sub-Nernstian response and an increase of the lower detection limit in FIG. 24. This behavior can result from the presence of a multiple of other elemental ions in the river water, e.g., K⁺ and Ca²⁺, that interfere with the Na⁺ detection. Nevertheless, the slope of the response curve improves with increasing α_{Na}_{⁺}, reaching 54.5±0.9 mV/dec when α_{Na}_{⁺} is above 30 mM, as a result of the reduced effect from the interfering ions at high analyte concentrations.

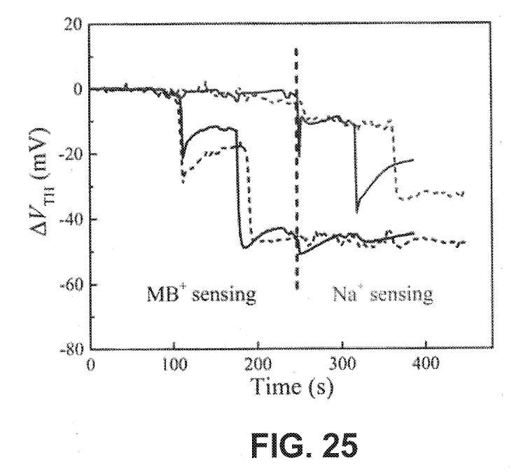

Multiplexed Analysis. The MB⁺-specific and Na⁺-specific SiNW-ISFET 62 sensors are integrated on the same chip, for a multiplexed analysis of molecular and elemental ions in one solution. The two MMMs are manually coated onto the sensors by drop casting. The hydrophobic MB⁺ will be extracted from the solution into the Na⁺-MMM 20A, giving rise to false response on the Na⁺-specific sensor. As a result, α_{MB}_{⁺} in the solution is kept below the threshold, i.e., 10 μM. As shown in FIG. 25, when the experiment is conducted with DI water, α_{MB}_{⁺} in the solution is first increased from 1 to 3 μM and then from 3 to 7 μM, the MB⁺-specific sensor shows ΔV_{TH} of 12.5 and 30.7 mV, respectively. This is in good agreement with previously demonstrated response shown in FIG. 21. With these changes, V_{TH} of the Na⁺-specific sensor remains unaltered. Afterwards, α_{Na}_{⁺} in the solution is increased from 100 to 300 μM and then from 300 μM to 1 mM, the corresponding ΔV_{TH} of the Na⁺-specific sensor is 9.4 and 13.0 mV; both are relatively small in comparison with the data obtained from the separate solution measurements as shown in FIG. 23B. This could be due to the use of a higher concentration of KCl, i.e., 10 mM, as the background electrolyte in this demonstration, since K⁺ is a strong interfering ion for the Na⁺-specific sensor. When the experiment was conducted with the river water, the same α_{MB}_{⁺} range as with DI water was applied since the MB⁺-specific sensor produces similar response curves in both water solutions. On the other hand, larger α_{Na}_{⁺} steps are used due to the performance deterioration of the Na⁺-specific sensor with the river water. As expected, the MB⁺-specific sensor shows ΔV_{TH} of 18.0 and 28.5 mV with the increase of α_{MB}_{⁺} first from 1 to 3 μM and then from 3 to 7 μM, respectively, while the Na⁺-specific sensor shows no detectable ΔV_{TH}. Afterwards, α_{Na}_{⁺} in the solution is increased from 30 to 300 μM and then from 300 μM to 3 mM, the corresponding ΔV_{TH} of the Na³⁰ -specific sensor is 10 and 22.5 mV.

Results demonstrate the possibility to operate the two types of sensors targeting, with high specificity, both molecular and elemental ions simultaneously in a complex sample.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Although the disclosure has been described with reference to certain embodiments, persons skilled in the art will recognize that changes may be made in form and detail without departing from the spirit and scope of the disclosed apparatus, systems and methods.

What is claimed is:

1. A chemically tunable metal-organic system comprising:
a. a supercontainer comprising one or more of:
i. an exo-cavity;
ii. an endo-cavity;
b. a substrate, wherein the substrate is combined with the supercontainer to form a scaffold; and
c. a reference probe,
wherein the system is constructed and arranged to detect ions.

2. The system of claim 1, wherein the system detects ions by measuring an electrical response.

3. The system of claim 1, wherein the substrate comprises:
i. a mixed-matrix membrane; or
ii. a solid sensing surface.

4. The system of claim 3, wherein the mixed-matrix membrane is a polymer matrix.

5. The system of claim 3, wherein the solid sensing surface is selected from the group consisting of oxide, nitride, and metal.

6. The system of claim 1, wherein the system measures electrical response using a solid-contact ion sensing electrode.

7. The system of claim 1, wherein the system measures electrical response using an ion-sensing electrode with an inner filling solution and an internal Ag/AgCl reference.

8. The system of claim 1, wherein the system measures electrical response using an ion-sensing electrode constructed on an ion-sensitive field-effect transistor gate terminal.

9. A device for sensing ions comprising:
a. a substrate, wherein the substrate consists of a metal organic super containers to form a scaffold;
b. an ion sensing electrode; and
c. a reference probe,
wherein, the device is configured to detect specific ions by measuring electrical response.

10. The device of claim 9, wherein the substrate is a mixed matrix membrane.

11. The device of claim 10, wherein the mixed matrix membrane comprises a variety of polymers.

12. The device of claim 11, wherein the polymer is selected from a group consisting of polyvinyl chloride, polydimethylsiloxane, poly(methyl methacrylate), Urushi, cellulose triacetate, poly aniline, poly urethane, siloprene, Poly-(vinyl chloride-co-vinyl acetate-co-hydroxypropyl acrylate), and Poly(vinyl chloride-co-vinyl acetate-co-vinyl alcohol).

13. The device of claim 9, wherein the substrate is a solid sensing surface.

14. The device of claim 13, wherein the solid sensing surface is selected from a group consisting of oxide, nitride, and metal.

15. The device of claim 9, further comprising an ion-sensitive field-effect transistor.

16. A system for sensing molecular ions comprising:
a. a substrate, wherein the substrate comprises a plurality of metal organic super containers designed to selectively bind a target molecular ion;
b. an ion sensing electrode; and
c. a reference electrode,
wherein the target molecular ion is detected by measuring an electrical response.

17. The system of claim 16, wherein the ion sensing electrode is a solid contact electrode and the substrate is placed on the electrode.

18. The system of claim 16, wherein the system is placed on an ion-sensitive field-effect transistor gate terminal to measure drain current change of the ion-sensitive field-effect transistor.

19. The system of claim 16, wherein the substrate is incorporated into the ion sensing electrode.

20. The system of claim 16, wherein the plurality of metal organic super containers are MOSC-II-Co molecules.
